(19) United States
(12) Patent Application Publication
(10) Pub. No.: US 2014/0214071 A1
(43) Pub. Date: Jul. 31, 2014

(54) EMBOLIC COIL DELIVERY SYSTEM AND METHOD OF USING SAME

(71) Applicant: NEURODYNAMICS, LLC., San Francisco, CA (US)

(72) Inventor: Jeffrey E. Thomas, Hillsborough, CA (US)

(73) Assignee: NEURODYNAMICS, LLC., San Francisco, CA (US)

(21) Appl. No.: 13/751,460

(22) Filed: Jan. 28, 2013

(51) Int. Cl. A61F 2/01

(52) U.S. Cl. CPC A61F 2/01
USPC 606/200

(57) ABSTRACT

A device and method of treating an aneurysm in a cranial artery of a patient includes providing a coil delivery system having a bypass catheter with a proximal end portion, a distal end portion, and an intermediate portion. The bypass catheter also includes a bypass lumen extending from the proximal end portion to the distal end portion and a coil delivery lumen that extends from the proximal end portion to a coil delivery opening in a side wall of the bypass catheter. The coil delivery system also includes an embolic coil or multiple coils capable of being deployed separately through the coil delivery opening and electrolytically detached, and at least one balloon disposed about an outer surface of the bypass catheter and capable of substantially preventing blood flow around the outer surface of the bypass catheter when inflated. The system may be used without coil embolization to provide endovascular bypass with segmental vessel occlusion during brain surgery such as craniotomy with clipping of a cerebral aneurysm.

### FIELD OF THE INVENTION

This application relates in general to an embolic coil delivery system and method for treating aneurysms in cerebral blood vessels. Specifically, this application relates to a catheter having an opening in its side wall so that an embolic coil may be deployed into a patient's aneurysm while the catheter transverses the neck of the aneurysm.

### BACKGROUND

Wide-necked aneurysms are generally not amenable to simple coil embolization because of the serious complications associated with prolapsed coil loops into the parent artery lumen or inadvertent loss of the entire coil into the normal brain circulation, causing stroke. Endovascular adjuncts, such as the balloon remodeling technique that temporarily makes the aneurysm neck smaller and the intracranial stents to isolate the aneurysm cavity and protect the parent artery lumen, have been utilized to make possible the coil embolization of wide-necked aneurysms.

It would be beneficial to develop a method of using coil embolization that did not require additional microcatheterization systems, which add to the complexity and intravascular bulk of the procedure, or powerful antiplatelet medication in order to decrease the risk of platelet-mediated vascular occlusion. It would also be beneficial to eliminate or greatly reduce the risk of endothelial disruption, arterial dissection, shearing, and in-stent stenosis that routinely occur with the use of intracranial stents.

### BRIEF SUMMARY

A device and method of treating an aneurysm in a cranial artery of a patient include providing a coil delivery system having a bypass catheter with a proximal end portion, a distal end portion, and an intermediate portion. The bypass catheter also includes a bypass lumen extending from the proximal end portion to the distal end portion of the bypass catheter and a coil delivery lumen that extends from the proximal end portion to a coil delivery opening in a side wall of the bypass catheter. The coil delivery system also includes an embolic coil capable of being deployed through the coil delivery opening and at least one balloon disposed about an outer surface of the bypass catheter and capable of substantially preventing blood flow around the outer surface of the bypass catheter when inflated. The coil delivery system may also include a coil delivery catheter that is configured to deploy the embolic coil through the coil delivery opening and into an aneurysm of a patient.

### DESCRIPTION OF THE DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 is a side view of a embolic coil delivery system deployed within the body.

FIG. 2 is a side view of another embodiment of a catheter for use in the embolic coil delivery system.

FIG. 3 is a side view of another embodiment of a catheter for use in the embolic coil delivery system.

FIG. 4 is a side view of another embodiment of a catheter for use in the embolic coil delivery system.

FIG. 5 is a side view of another embodiment of a catheter for use in the embolic coil delivery system.

FIG. 6 is a side view of another embodiment of the embolic coil delivery system deployed within the body.

### DETAILED DESCRIPTION

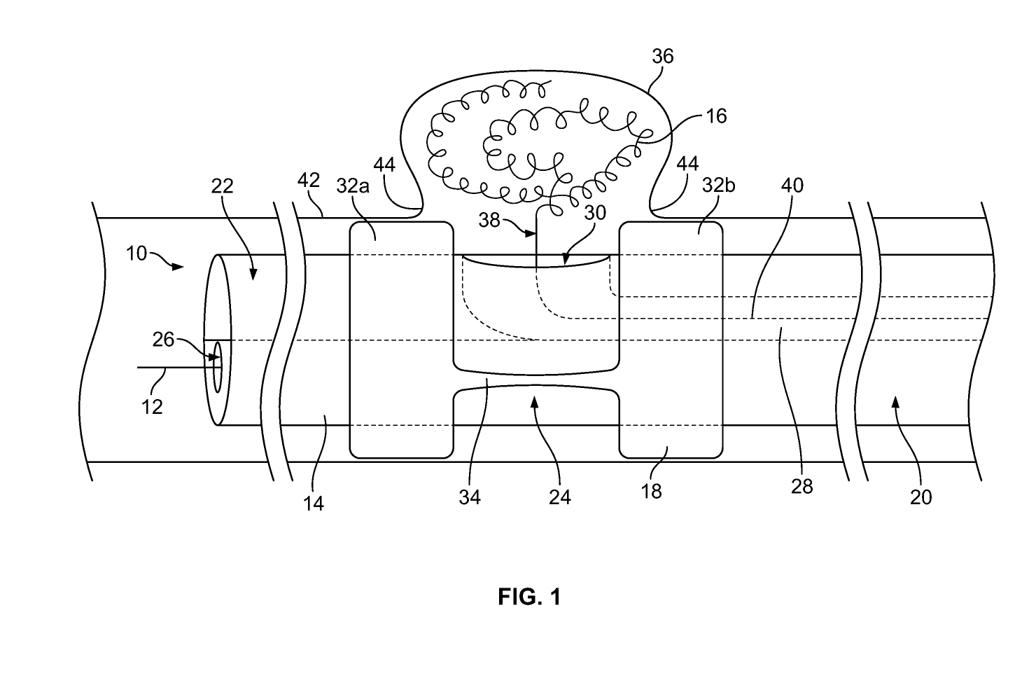

FIG. 1 is a side view of one embodiment of an embolic coil delivery system (“the delivery system”) 10 deployed within a patient's body. The delivery system 10 may be used to treat wide-necked aneurysms in a patient's brain and may generally include a guidewire 12, a bypass catheter 14, a embolic coil 16, and, optionally, an inflatable balloon 18. Each of these elements will be discussed in more detail below.

As discussed above, the delivery system 10 may include one or more guidewires. The primary guidewire 12 may be coated with a hydrophilic substance and may be used for initial placement of the delivery system 10. The primary guidewire 12 may have a length from about 180 cm to about 190 cm.

The bypass catheter 14 includes a tubular body with a proximal end portion 20, a distal end portion 22, and an intermediate portion 24. The bypass catheter 14 also includes a bypass lumen 26 extending the length of the bypass catheter 14 and a coil delivery lumen 28 extending from a coil opening 30 in the sidewall of the intermediate portion 24 of the bypass catheter 14 to a coil delivery portal (not shown) in the proximal end portion 20 of the bypass catheter 14.

For intracranial applications, the bypass catheter 14 may have a length from 150 cm to about 300 cm and may have an external diameter from 0.62 mm to 0.95 mm. However, it should be appreciated that catheters of any suitable dimensions may be used. The bypass catheter 14 may all be made of a metallic material, such as stainless steel or titanium, a plastic or polymeric material, or a combination of the two. Other suitable materials are also contemplated.

The bypass catheter 14 may also include a balloon 32 disposed about its outer surface. As shown in FIG. 1, in one embodiment, the bypass catheter 14 includes two balloons 32a and 32b disposed on either side of the coil opening 30. In this embodiment, the distal balloon 32a and the proximal balloon 32b may be inflated through a single inflation lumen (not shown) by providing continuity 34 between the proximal and distal balloons.

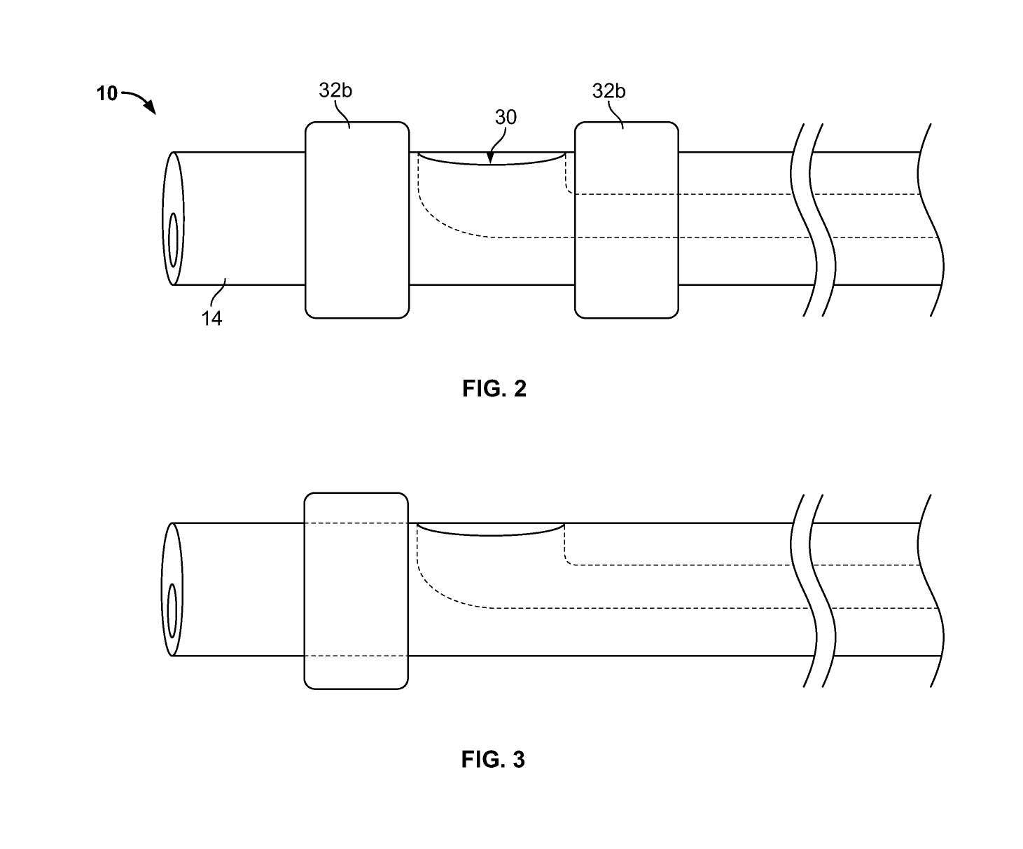

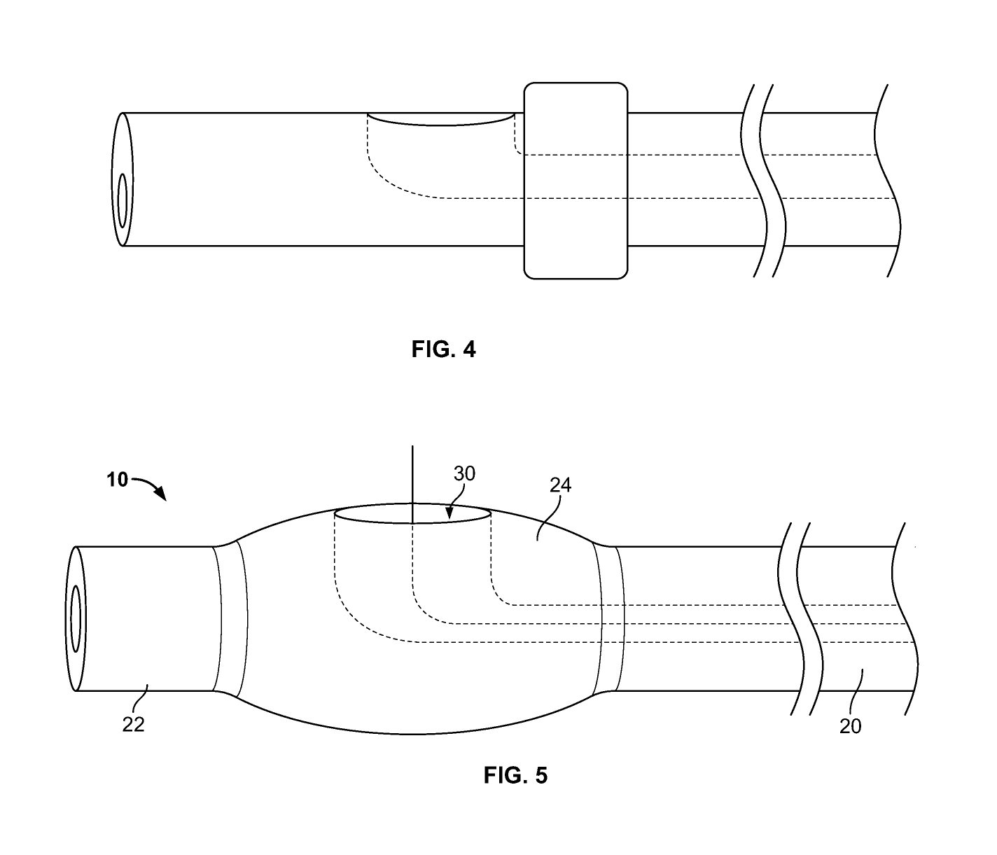

FIGS. 2-5 show alternative embodiments of the delivery system 10. As shown in FIG. 2, the balloons 32a and 32b may be separate, rather than in continuity with one another, as shown in FIG. 1. In another embodiment, as shown in FIGS. 3-4, the delivery system 10 may only include one balloon 32, disposed distally of the coil delivery opening 30 (FIG. 3) or proximally of the coil delivery opening 30 (FIG. 4). In another embodiment, the delivery system 10 may include no balloon. In yet another embodiment, the intermediate portion 24 of the bypass catheter 14 may be focally thickened (FIG. 5) in order to effectively narrow the neck 44 of the aneurysm 36 without a balloon.

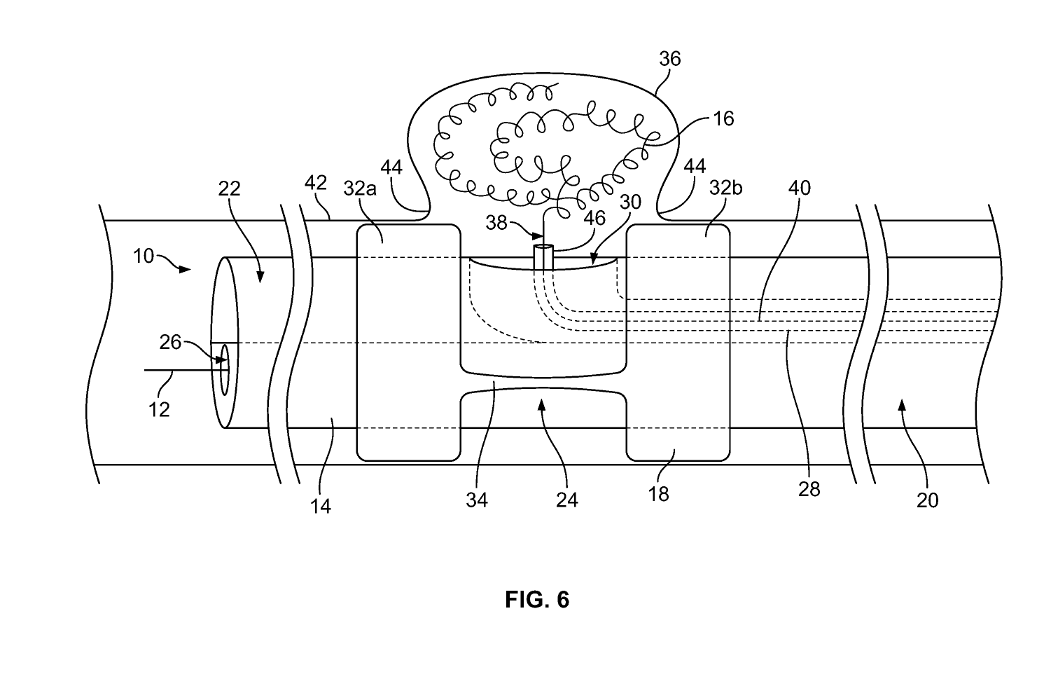

Referring again to FIG. 1, the embolic coil 16 may include any detachable embolic coil that is capable of expanding to fill the anuerysm 36. Embolic coils may be made of any suitable material, and include but are not limited to platinum, platinum/tungsten and Nitinol. In one embodiment, the embolic coil 16 may include a straight segment 38 at its proximal end. The straight segment facilitates placement of the embolic coil 16 into the aneurysm 36 and provides a supportive surface for a pusher wire 40 to advance the embolic coil 16 through the coil delivery lumen 28. In FIG. 6, the delivery system 10 may also include a coil delivery catheter 46 (or microcatheter) that is configured to deploy the embolic coil 16 through the coil delivery opening 30 and into an aneurysm 36 of a patient.

FIG. 1 shows the delivery system 10 in relation to an aneurysm 36 in the vessel wall 42 of a patient's cranial artery. In use, the bypass catheter 14 is advanced through the patient's vessel over the guidewire 12 so that the distal end portion 22 of the bypass catheter 14 is disposed beyond the neck 44 of the aneurysm 36 and the coil opening 30 in the side wall of the bypass catheter 14 is placed within the neck 44 of the aneurysm 36. The distal balloon 32a and the proximal balloon 32b are placed on either side of the neck 44 of the aneurysm 36 and are inflated. Any suitable imaging techniques may be used to position the coil opening 30 and the balloons 32a and 32b at and around the neck 44 of the aneurysm 36. These techniques include, but are not limited to, the use of fluoroscopy combined with radiopaque markers (not shown) on the bypass catheter itself. Once in place, the bypass catheter 14 may need to be rotated in order to align the coil opening 30 with the neck 44 of the aneurysm 36.

Once in place, the balloons 32a and 32b are inflated and effectively narrow the width of the neck, lessening the likelihood that the embolic coil, when deployed, will migrate out of the aneurysm 36 and back into the coil delivery lumen 28 or into the vessel itself. When inflated, the balloons 32a and 32b press against the vessel wall 42 and prevent blood flow around the bypass catheter 14.

The bypass catheter 14, however, may be used to delivery arterialized blood to the brain through the bypass lumen 26, despite the inflation of the balloons 32a and 32b. By using the bypass catheter 14 to deliver blood to the brain, the amount of time that the physician has to deploy and effectively implant the embolic coil 16 within the aneurysm 36 is increased dramatically.

Generally, the bypass catheter 14 may extend considerably (up to 10 cm) farther beyond neck 44 of the aneurysm 36 in order to effectuate the bypass method. In addition to the arterialized blood, thrombolytic agents, cold plasma, and saline may be administered past the aneurysm in the manner described above. The cold plasma may be used to create regionalized hypothermia, extending the time the surgeon may have to effectively deploy the embolic coil 16.

Once in place, the delivery system 10 may be used to introduce the embolic coil 16 into the aneurysm 36. The embolic coil 16 is delivered through the coil delivery lumen 28 and through the coil delivery opening 30. In one embodiment, the coil 16 may be modified at its proximal end by adding a relatively straight segment 38 adjacent to the pusher wire 40. The straight segment 38 facilitates the perpendicular delivery of the embolic coil into the aneurysm 36 and results in increased penetration and embedding of the embolic coil 16 into the aneurysm 36.

The ultimate stability of the embolic coil 16 within the wide-necked aneurysm 36 is addressed by the bypass feature of the delivery system 10, continuously providing blood and other medicaments to the brain. The stability depends in part upon the clotting of blood throughout the embolic coil 16 and, while the stabilizing influence of the balloons 32, the bypass catheter 14 or its widened counterpart, must eventually be withdrawn, the longer it remains in place, the more likely it will be that the embolic coil 16 will stabilize within the aneurysm 36. Generally, the bypass catheter 14 and balloons 32 should remain in place for about two to five minutes or until it can be determined that sufficient clotting has taken place within the aneurysm 36. However, because of the use of the bypass catheter 14, the delivery system 10 can remain in place for as long as necessary to effectuate clotting around the embolic coil 16. Multiple embolic coils may also be used to occlude the aneurysm, in the usual manner of endovascular coil embolization.

In addition, the delivery system 10 may be used to treat an intraoperative rupture of the aneurysm 36. By using the bypass catheter 14 to delivery arterialized blood to the brain and the balloons 32 to occlude the vessel, not only is catastrophic hemorrhage, such as intraoperative rupture, immediately stopped, but the bypass feature allows the balloons 32 to create a segmental occlusion of the vessel for prolonged periods of time, increasing the likelihood that hemostasis will be effective. This type of segmental vessel occlusion may also be used in open surgery in order to provide the surgeon with additional time to control an intraoperative rupture. Therefore, the delivery system 10, in its capacity of creating a segmental vessel occlusion while preserving distal blood flow, can also be used as an adjunct to aneurysm clipping surgery or any neurosurgery requiring temporary occlusion of a blood vessel.

While example methods and compositions have been illustrated by describing examples, and while the examples have been described in considerable detail, it is not the intention of the applicant to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention is not limited to the specific details, the representative revascularization catheter systems, and illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

1. A embolic coil delivery system comprising:
a bypass catheter comprising a proximal end portion, a distal end portion, and an intermediate portion, wherein the bypass catheter also comprises a bypass lumen that extends from the proximal end portion to the distal end portion and a coil delivery lumen that extends from the proximal end portion to a coil delivery opening disposed in a side wall of the bypass catheter; and
an embolic coil capable of being deployed through the coil delivery opening.

2. The embolic coil delivery system of claim 1, wherein the embolic coil delivery system further comprises at least a first balloon disposed about an outer surface of the bypass catheter; and wherein the at least one balloon is capable of substantially preventing the flow of blood around the outer surface of the bypass catheter when inflated.

3. The embolic coil delivery system of claim 2, wherein the first balloon is disposed distally of the coil delivery opening.

4. The embolic coil delivery system of claim 2, wherein the first balloon is disposed proximally of the coil delivery opening.

5. The embolic coil delivery system of claim 4, wherein the embolic coil delivery system further comprises a second balloon and wherein the second balloon is disposed distally of the coil delivery opening.

6. The embolic coil delivery system of claim 5, wherein the embolic coil delivery system further comprises an inflation lumen and wherein the first balloon and the second balloon are capable of being inflated through the inflation lumen.

7. The embolic coil delivery system of claim 5, wherein the first balloon and the second balloon are configured to be inflated through separate inflation lumens.

8. The embolic coil delivery system of claim 1, wherein the embolic coil delivery system further comprises a coil delivery catheter capable of deploying the embolic coil through the coil delivery opening.

9. The embolic coil delivery system of claim 1, wherein the intermediate portion of the bypass catheter is focally thickened.

10. A method of treating an aneurysm in a cranial artery of a patient comprising the steps of:
providing a coil delivery system comprising:
a bypass catheter comprising a proximal end portion, a distal end portion, and an intermediate portion, wherein the bypass catheter also comprises a bypass lumen that extends from the proximal end portion to the distal end portion and a coil delivery lumen that extends from the proximal end portion to a coil delivery opening in a side wall of the bypass catheter;
at least one embolic coil capable of being deployed through the coil delivery opening; and
positioning the coil delivery system within a cranial artery of a patient so that the coil delivery opening is disposed at a neck of the aneurysm;
providing arterialized blood through the bypass lumen; and
deploying the embolic coil into the aneurysm.

11. The method of claim 10, wherein the coil delivery system further comprises at least a first balloon disposed about an outer surface of the bypass catheter.

12. The method of claim 11, wherein the method further comprises the step of inflating the first balloon and substantially preventing the flow of blood around the outer surface of the bypass catheter when inflated.

13. The method of claim 10, wherein the first balloon is disposed distally of the coil delivery opening.

14. The method of claim 10, wherein the first balloon is disposed proximally of the coil delivery opening.

15. The method of claim 14, wherein the coil delivery system further comprises a second balloon disposed about the outer surface of the bypass catheter and wherein the second balloon is disposed distally of the coil delivery opening.

16. The method of claim 15, wherein the embolic coil delivery system further comprises an inflation lumen and wherein the first balloon and the second balloon are capable of being inflated through the inflation lumen.

17. The method of claim 15, wherein the first balloon and the second balloon are configured to be inflated through separate inflation lumens.

18. The method of claim 10, wherein the embolic coil delivery system further comprises a coil delivery catheter capable of deploying the embolic coil through the coil delivery opening.
